(19) United States
(12) Patent Application Publication
(10) Pub. No.: US 2007/0224200 A1
(43) Pub. Date: Sep. 27, 2007

(54) Gm3 Synthase as a Therapeutic Target in Microvascular Complications of Diabetes

(76) Inventors: Samer Elbawab, Paris (FR); Elodie Masson, Villeurbanne (FR); Daniel Ruggiero, Sainte Consorce (FR); Nicolas Wiernsperger, Orlieanas (FR); Michel Lagarde, Decines (FR); Lysiane Troncy, Lamure-sur-Azergues (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/579,725

(22) PCT Filed: Apr. 7, 2005
(86) PCT No.: PCT/EP2005/03647

§ 371 Date: Nov. 6, 2006

(30) Foreign Application Priority Data
May 7, 2004 (FR) 0404971
Dec. 17, 2004 (FR) 0413530

(51) Int. Cl. A61K 39/395; A61K 31/00; A61K 31/7105; C12Q 1/02; A61K 38/43

(52) U.S. Cl. 424/146.1; 424/158.1; 424/94.1; 435/29; 514/1; 514/44

(57) ABSTRACT

The present invention relates to the use of an inhibitor of the expression or activity of the GM3 synthase gene for the treatment of microvascular complications of diabetes, and to a method of screening compounds useful in the treatment and/or prevention of these complications.

The invention relates to the use of GM3 synthase inhibitors for treating microvascular complications of diabetes, and to a method of screening inhibitors of this enzyme.

Microangiopathy is a chronic complication of diabetes which is characterised by structural and functional changes to the microvessels. The retina and the kidney are the two main targets of the pathological condition, leading to diabetic retinopathy and diabetic nephropathy.

Diabetic retinopathy is the second cause of blindness in developed countries. After about twenty years of the disease, almost all patients with type 1 diabetes and more than 60% of patients with type 2 diabetes suffer from this microvascular complication (Fong et al., 2003). The capillaries undergo progressive structural changes, such as a thickening of the basal membrane and the specific loss of pericytes, and subsequent modifications of the proliferation and function of the endothelial cells. These changes, combined with ischaemia, damage the microvessel wall and promote an excessive capillary permeability, leading to oedemas, microaneurisms and haemorrhages that threaten eyesight (Forrester et al., 1997).

Nephropathy affects 50 to 60% of patients with a 20- to 30-year history of diabetes. It is considered to be a major cause of mortality in these patients (Krolewski et al., 1997). One of the main characteristics of the pathological condition is glomerular enlargement, which is a consequence of thickening of the basal membrane and expansion of the mesangium due to hypertrophy of the mesangial cells in arrested growth and due to the accumulation of proteins of the extra-cellular matrix. These events, combined with haemodynamic deficiencies, induce glomerular sclerosis, glomerular filtration or a changed level of glomerular filtration and microalbuminuria, leading to severe renal insufficiency (Wolf et al., 2000).

The risk of a diabetic subject developing nephropathy is generally evaluated by checking the microalbuminuria. Preventive or therapeutic means currently used for delaying the appearance and/or progression of diabetic nephropathy include the monitoring of glycaemia, the administration of antihypertensives, especially angiotensin converting enzyme inhibitors, the adoption of a low-protein diet or the administration of hypolipidaemics such as statins (Rippin et al., 2004).

Given their impact in terms of public health and economics, the identification of novel ways of preventing and treating microvascular complications of diabetes constitutes a major therapeutic challenge.

Although the cellular and molecular basis of the pathogenesis of diabetic retinopathy and diabetic nephropathy is not completely understood, the regulation of cell proliferation and the cell-cell and cell-matrix interactions seem to play an important role. A number of biochemical hypotheses have been proposed to explain the mechanisms involved in the development of microvascular complications of diabetes, especially the formation of advanced glycation end products (AGE) (Singh et al., 2001).

Reducing sugars such as glucose react non-enzymatically with the amino groups of proteins, lipids and nucleic acids via a series of reactions that form Schiff bases and Amadori products, ultimately producing AGE. Glycation, which is dependent on glucose concentration, is increased in diabetes. It occurs preferentially with long-lived proteins exposed to the blood glucose, such as the proteins of the extracellular matrix or the circulating proteins, thereby modifying their structure and their function.

In addition, AGE can bind membrane receptors to induce cellular responses via the generation of oxidative stress (Lal et al., 2002; Schmidt et al., 1994), the activation of nuclear factor κB (Singh et al., 2001; Schmidt et al., 1994) and the expression of different genes such as proinflammatory cytokines or adhesion molecules (Hofmann et al., 1999; Schmidt et al., 1995).

All these modifications have important biological effects which can explain many of the changes observed in microvascular complications of diabetes, especially the increase in vascular permeability, the increase in the production and rigidity of the extracellular matrix, and the change in cell-matrix interactions and cell growth (Stitt et al., 2003). In fact, many in vivo and in vitro studies have suggested that AGE are involved in the development of the retinopathy and nephropathy associated with diabetes (Stitt et al., 2003; Wautier et al., 2001).

Gangliosides are glycosphingolipids that are concentrated in microdomains of the plasmic membrane and characterised by the presence of sialic acid in their structure. Successive sialylations of lactosylceramide produce monosialoganglioside (GM3), disialoganglioside (GD3) and trisialoganglioside (GT3). These gangliosides are then converted by the sequential action of glycosyltransferases and sialyltransferases to more complex gangliosides, forming the a, b and c series, respectively (Van Echten et al., 1993). Gangliosides are known to play a major role in cell-cell and cell-matrix recognition by interaction with adhesion receptors such as integrins or matrix proteins (collagen and fibronectin), or with other glycosphingolipids. Also, gangliosides, particularly those of the a series, have been implicated in the regulation of cell proliferation via modulation of the activity of different growth factors (Hakomori et al., 1990).

It has been reported that AGE induce modifications to the metabolism of glycosphingolipids in pericytes and. endothelial cells of retinal microvessels (Natalizio et al., 2001). These changes are accompanied especially by an increase in GM3 synthase activation (A. Daleme-Natalizio, doctoral thesis at the Institut National des Sciences Appliquées de Lyon, 8 Feb. 2002).

The inventors have now shown that gangliosides are involved in the AGE-mediated effects that lead to the pathological conditions DR and DN. The inventors have thus demonstrated that the inhibition by AGE of the proliferation of retinal pericytes and renal mesangial cells—the two cell types involved in diabetic retinopathy and diabetic nephropathy, respectively—is at least partly based on the increase in GM3 synthase activity and the accumulation of series a gangliosides. An increase in GM3 synthase activity has moreover been observed in a diabetic mouse model to which AGE have been given. These results identify GM3 synthase and series a gangliosides as targets for treating microvascular complications of diabetes.

### Definitions

The expression “microvascular complication of diabetes” denotes a chronic complication of type I or II diabetes that is characterised by structural and functional changes in the microvessels. These complications mainly include diabetic retinopathy, diabetic nephropathy and diabetic neuropathy. Peripheral neuropathy affects the nerves of the body extremities, loss of sensation in the feet being one of its most widespread forms. Discomfort and pain (paraesthesia and hyperaesthesia) are also very common and debilitating symptoms. This neuropathy can cause foot ulcers and serious tissue damage that may necessitate amputation.

Within the framework of the present patent application, the expression “GM3 synthase” denotes the enzyme lactosylceramide α2,3-sialyltransferase (EC 2.4.99.9), which catalyses the transfer of a sialic acid residue from a sialic acid donor to a 3-hydroxyl group of a galactose residue of a sialic acid acceptor. Preferentially, the sialic acid donor is CMP-N-acetylneuramic acid and the sialic acid acceptor is a galactose residue of a glycolipid such as lactosylceramide (LacCer). The catalysed reaction may be CMP-N-acetylneuraminate+β-D-galactosyl-1,4-β-D-glucosylceramide=Cmp+α-N-acetylneuraminyl-2,3-β-D-galactosyl-1,4-β-D-glucosylceramide. Preferably, the GM3 synthase according to the invention is a human GM3 synthase or a GM3 synthase of a non-human mammal such as a rodent (e.g. rat or mouse), a feline, a canine, a primate (monkey), etc. For example, the genes coding for human and murine GM3 synthase have been respectively deposited in the Genbank database under the accession numbers NM_{—}003896 (SEQ ID No: 1) and NM_{—}011375 (SEQ ID No: 3). The corresponding amino acid sequences are described in the sequences SEQ ID No: 2 and SEQ ID No: 4, respectively.

Within the framework of the present patent application, a “GM3 synthase inhibitor” denotes a compound which: (i) inhibits the activity and/or expression of GM3 synthase in vitro and/or in vivo; and/or (ii) blocks the transfer of a sialic acid residue from a sialic acid donor to a 3-hydroxyl group of a galactose residue of a sialic acid acceptor, especially to form ganglioside GM3; and/or (iii) blocks the intracellular synthesis of ganglioside GM3. The inhibition or blocking can be partial or total.

“Ganglioside” is understood as meaning a glycosphingolipid comprising one or more sialic acid residues. More specifically, “series a gangliosides” denote gangliosides carrying only one sialic acid residue on the galactose of the lactosylceramide. Series a gangliosides include the compounds GM3 (α-N-acetyl-neuraminyl-2,3-β-D-galactosyl-1,4-β-D-glucosylceramide), GM2, GM1, GD1a and GT1a (cf. FIG. 2).

### Therapeutic Application

The inventors have demonstrated that the advanced glycation end products (AGE) involved in the development of microvascular complications of diabetes mediate their effects via an increase in the activity of the GM3 synthase enzyme.

The invention therefore proposes a method of treating a microvascular complication of diabetes wherein an inhibitor of the expression or activity of the GM3 synthase gene is administered to the patient.

The invention further relates to the use of an inhibitor of the expression or activity of the GM3 synthase gene for the manufacture of a drug intended for the treatment of a microvascular complication of diabetes.

Preferably, the microvascular complication of diabetes is selected from the group comprising diabetic retinopathy, diabetic nephropathy and diabetic neuropathy. Particularly preferably, the microvascular complication of diabetes is diabetic nephropathy.

Within the framework of the invention, the expression “treatment” denotes the preventive or curative treatment of a disease, i.e. the act of reversing, slowing down or inhibiting the progression, or preventing the development, of a disease or of one or more symptoms attached to this disease.

The expression “patient” denotes a human or a non-human mammal, such as a mouse, rat, dog, cat, pig or monkey, that is affected or liable to be affected by a microvascular complication of diabetes. Preferably, a patient in terms of the invention is a subject in which diabetes has been detected.

Preferably, the inhibitor is a specific inhibitor of the expression or activity of the GM3 synthase gene, i.e. an inhibitor substantially devoid of an effect on genes or proteins other than GM3 synthase.

In a first embodiment, the method or use according to the invention employs an inhibitor of the expression of the GM3 synthase gene and/or protein. Such an inhibitor can inhibit or repress the transcription of the gene and/or the translation of the transcript messenger (mRNA). Those skilled in the art are capable of choosing the most suitable strategy for this purpose.

An antisense strategy can be used to inhibit GM3 synthase expression. This approach can utilise e.g. antisense nucleic acids or ribozymes which block the transcription of a specific mRNA, either by masking the mRNA with an antisense nucleic acid, or by cleaving the mRNA with a ribozyme. In the context of the present invention, “antisense” broadly includes RNA-RNA interactions, RNA-DNA interactions, ribozymes, interfering RNAs, aptamers, and inhibition mediated by RNAseH. An antisense therapy generally employs a vector, such as a viral vector, which carries the antisense sequence, the inhibition then being generally stable since the vector will integrate into the genome. It is also possible to use antisense oligonucleotides, which bring about a transitory inhibition of expression. A general presentation of the antisense technique can be found in “Antisense DNA and RNA” (Cold Spring Harbor Laboratory, D. Melton, ed., 1988).

Preferably, the inhibitor of the expression of the GM3 synthase gene and/or protein is therefore selected from the group comprising antisense nucleic acids, ribozymes, interfering RNAs and aptamers.

An “antisense nucleic acid” or an “antisense oligonucleotide” is a single-stranded nucleic acid molecule which, when it hybridises under cytoplasmic conditions with a complementary DNA or RNA molecule, inhibits the function of the latter. Antisense nucleic acids can be encoded by a recombinant gene for expression in a cell (cf., for example, U.S. Pat. No. 5,814,500 and 5,811,234), or they can be prepared by synthesis (cf., for example, U.S. Pat. Nos. 5,780,607). Antisense nucleic acids of GM3 synthase can be designed to hybridise specifically with a homologous sequence coding for a GM3 synthase, e.g. to hybridise specifically with the human GM3 synthase sequence shown in SEQ ID No: 1 or the murine GM3 synthase sequence shown in SEQ ID No: 3.

A “sequence capable of hybridising specifically with a nucleic acid sequence” denotes a sequence that hybridises with a reference nucleic acid sequence under highly stringent conditions (Sambrook et al., 1989). The parameters defining the stringency conditions depend on the temperature at which 50% of the matching strands separate (Tm), and on the ionic strength. For sequences comprising more than 30 bases, Tm is defined by the following equation: Tm=81.5+0.41(% G+C)+16.6 log(cation concentration)−0.63(% formamide)−(600/number of bases) (Sambrook et al., 1989). For sequences shorter than 30 bases, Tm is defined by the following equation: Tm=4(G+C)+2(A+T). Under appropriate stringency conditions where the non-specific sequences do not hybridise, the hybridisation temperature can preferably be 5 to 10° C. below Tm and the hybridisation buffers used are preferably solutions of high ionic strength, such as a 6×SSC solution. For example, highly stringent hybridisation conditions correspond to Tm and to ionic conditions such as those obtained with a solution containing 50% formamide and 5× or 6×SCC (0.15 M NaCl, 0.015 M sodium citrate).

The antisense nucleic acid according to the invention can be used as such, for example after injection into the human or animal, to induce protection or treat a microvascular complication of diabetes. In particular, they can be injected in the form of naked DNA according to the technique described in international patent application WO 90/11092. They can also be administered in the form of a complex with e.g. dextran-DEAE (Pagano et al., 1967), nuclear proteins (Kaneda et al., 1989) or lipids (Felgner et al., 1987), in the form of liposomes (Fraley et al., 1980) or by other similar methods.

Preferably, the nucleic acid sequences form part of a vector. The use of a vector makes it possible to improve the administration of the nucleic acid to the cells to be treated, and also improves the stability in these cells, affording a prolonged therapeutic effect.

The term “vector” denotes the vehicle via which a DNA or RNA sequence can be introduced into a host cell so as to transform the host and obtain the expression (i.e. the transcription and translation) of the sequence which has been introduced. Vectors include plasmids, phages, viruses, etc.

“Ribozymes” are RNA molecules that have the capacity specifically to cleave other single-stranded RNA molecules in a manner fairly analogous to DNA restriction endonucleases. Ribozymes were discovered by demonstrating that certain mRNAs have the capacity to cleave their own introns. By modifying the nucleotide sequence of these ribozymes, it is possible to produce molecules that recognise specific nucleotide sequences in RNA molecules and cleave them (Cech, 1989). Because of this specificity, only mRNAs that have a particular sequence are inactivated.

Reversible inhibition of GM3 synthase transcription can also be achieved using interfering RNAs. The technique of RNA interference (RNAi) prevents the expression of genes by using small RNA molecules such as “small interfering RNAs” (siRNAs). This technique benefits from the fact that RNA interference is a natural biological mechanism of gene extinction in the majority of cells of numerbus living organisms, from plants to insects and up to mammals (Sharp, 2001). RNA interference prevents the production of a functional protein from a gene by leading to the destruction of the intermediate mRNA (Bass, 2000; Sharp, 2001). The siRNAs can be used in naked form or incorporated into a vector. Preferably, an interfering RNA that blocks GM3 synthase transcription can have the sequence GGGUUAUUCUGAACAUGUUtt (SEQ ID No: 5).

Aptamers can also be used to inhibit GM3 synthase transcription. Aptamers are oligonucleotide sequences that have the capacity to recognise virtually any class of target molecules with a high affinity and specificity. Such ligands can be isolated from a random sequence library by a screening method called SELEX (Systematic Evolution of Ligands by EXponential enrichment), as described in Tuerk and Gold (1990). The random sequence library can be obtained by DNA synthesis by means of combinatorial chemistry. In such a library, each member is a linear oligomer (optionally chemically modified) corresponding to a unique sequence. The possible modifications, applications and advantages of this class of molecules have been reviewed by Jayasena (1999).

In another embodiment, the method or use according to the invention involves using an inhibitor of the activity of the GM3 synthase protein. Inhibitors of GM3 synthase activity can easily be identified by screening methods, including cellular or biochemical tests in vitro, as described in the present patent application. An inhibitor can be of a peptide nature, a peptidomimetic or a non-peptide mimic (Rubin-Carrez, 2000), such as a small organic molecule capable of interfering with the enzymatic activity of GM3 synthase, for example by blocking or reducing the transfer of a sialic acid group from a donor to a sialic acid acceptor, and/or by blocking or reducing GM3 synthesis.

The GM3 synthase inhibitor can also be an antibody, particularly an inhibitor directed against the human GM3 synthase shown in the sequence SEQ ID No: 2 or the murine GM3 synthase shown in the sequence SEQ ID No: 4. Said antibodies can be polyclonal or monoclonal antibodies or fragments thereof, or chimeric antibodies, especially those which are humanised or immunoconjugated.

Polyclonal antibodies can be obtained by the customary procedures from the serum of an animal immunised against a protein. For example, the antigen used can be an appropriate peptide complex such as a complex of GM3 synthase coupled via a reactive residue to a protein (such as keyhole limpet haemocyanin, KLH) or another peptide. Rabbits are immunised with the equivalent of 1 mg of the peptide antigen according to the procedure described by Benoit et al. (1982). At four-week intervals the animals are treated with 200 μg injections of antigen and bled 10 to 14 days later. After the third injection, the antiserum is examined in order to determine its capacity to bind to the iodine-radiolabelled peptide antigen, prepared by the chloramine-T method, and is then purified by chromatography on a carboxymethyl cellulose (CMC) ion exchange column. The antibody molecules are then collected from the mammals and isolated to the desired concentration by the methods well known to those skilled in the art, for example by using DEAE Sephadex to obtain the IgG fraction. To increase the specificity of the polyclonal serum, the antibodies can be purified by immunoaffinity chromatography using immunising polypeptides in the solid phase. The antibody is brought into contact with the immunising polypeptide in the solid phase for a sufficient time to cause the polypeptide to undergo an immune reaction with the antibody molecule in order to form an immunological complex in the solid phase.

Monoclonal antibodies can be obtained by the conventional lymphocyte fusion and hybridoma culture method described by Köhler and Milstein (1975). Other methods of preparing monoclonal antibodies are also known (Harlow et al., 1988). Monoclonal antibodies can be prepared by immunising a mammal (for example a mouse, rat or rabbit, or even a human, etc.) and using the lymphocyte fusion technique to produce hybridomas (Köhler and Milstein, 1975). There are alternatives to this customary technique. It is possible, for example, to produce monoclonal antibodies by expressing a nucleic acid cloned from a hybridoma. Antibodies can also be produced by the phage display technique by introducing antibody cDNAs into vectors, the latter typically being filamentous phages with V gene libraries on the surface of the phage (e.g. fUSE5 for E. coli, Scott and Smith, 1990). Protocols for constructing these antibody libraries are described in Marks et al. (1991).

The antibodies or antibody fragments of the invention can be e.g. chimeric antibodies, humanised antibodies or Fab and F(ab′)₂ fragments. They can also take the form of immunoconjugates or labelled antibodies.

Aptamers constitute a class of molecules that represent an alternative to antibodies in terms of molecular recognition.

The inhibitors of the expression or activity of GM3 synthase can be formulated with one or more pharmaceutically acceptable excipients. As described earlier, these inhibitors can be a chemically synthesised compound, an antisense or interfering RNA or an anti-GM3 synthase antibody.

“Excipient” or “pharmaceutically acceptable vehicle” is understood as meaning any solvent, dispersion medium, absorption retarder, etc. that does not produce a secondary reaction, for example an allergic reaction, in humans or animals.

The dosage naturally depends on the active substance in question, the mode of administration, the therapeutic indication and the age and state of the patient. The dose of protein or antibody is preferably 0.1 to 250 mg/kg per day and particularly preferably 1 to 100 mg/kg per day. When the pharmaceutical compositions comprise nucleic acids, the doses of nucleic acid (sequence or vector) to be administered are also adapted in particular according to the mode of administration, the targeted pathological condition and the duration of the treatment. In general, if recombinant viruses are used, these are formulated and administered as doses of about 10⁴ to 10¹⁴ pfu/ml and preferably of 10⁶ to 10¹⁰ pfu/ml. The term “pfu” (plaque forming unit) corresponds to the infectivity of a viral solution and can be determined by infecting an appropriate cell culture and measuring the number of plaques of infected cells, generally after 48 hours. The techniques for determining the pfu titre of a viral solution are amply described in the literature.

If parenteral administration is envisaged, more particularly by injection, the compositions of the invention comprising the active principle(s) take the form of injectable solutions and suspensions packaged in ampoules or bottles for slow perfusion. Injection can be effected especially by the subcutaneous, intramuscular or intravenous route.

In the case of oral administration, the compositions of the invention take the form of gelatine capsules, effervescent tablets, coated or uncoated tablets, sachets, dragees, ampoules or solutions to be taken orally, microgranules or prolonged release forms.

The forms for parenteral administration are obtained in conventional manner by mixing the active principle(s) with buffers, stabilisers, preservatives, solubilisers, isotonic agents and suspending agents. Using the known techniques, these mixtures are subsequently sterilised and then packaged in the form of intravenous injections.

The buffers used by those skilled in the art may be those based on organic phosphate salts.

Examples of suspending agents include methyl cellulose, hydroxyethyl cellulose, hydroxypropyl cellulose, acacia and sodium carboxymethyl cellulose.

Moreover, stabilisers useful according to the invention are sodium sulfite and sodium metabisulfite, while preservatives which may be mentioned are sodium p-hydroxybenzoate, sorbic acid, cresol and chlorocresol. To prepare an oral solution or suspension, the active principles are dissolved or suspended in an appropriate vehicle with a dispersant, a humectant, a suspending agent (e.g. polyvinylpyrrolidone), a preservative (such as methylparaben or propylparaben), a taste corrector or a colourant.

To prepare microcapsules, the active principles are combined with appropriate diluents, appropriate stabilisers, agents promoting the prolonged release of the active substances, or any other type of additive to form a central core, which is then coated with an appropriate polymer (e.g. a water-soluble resin or a water-insoluble resin). The techniques known to those skilled in the art will be used for this purpose.

The resulting microcapsules are then optionally formulated into appropriate dosage units.

Administration by the ocular route can also be envisaged.

In that case the pharmaceutical composition of the invention takes the form of an ophthalmic composition for local administration to the eye, for example an eye lotion or ophthalmic cream.

The inhibitors can also be formulated as liposomes. Liposomes are formed from phospholipids, which are dispersed in an aqueous medium and spontaneously form multilamellar concentric bilayer vesicles. These vesicles generally have a diameter of 25 nm to 4 μm and can be sonicated, resulting in the formation of smaller unilamellar vesicles, with a diameter of 200 to 500 Å, containing an aqueous solution in their core.

Liposomes can be particularly advantageous for the administration of an active principle to a precise cellular or tissular target. This can be done by chemically coupling the lipids to targeting molecules, such as targeting peptides (for example hormones), or antibodies.

### Screening Method

The invention further relates to an in vitro method of screening or identifying compounds useful in the treatment and/or prevention of microvascular complications of diabetes, wherein the capacity of at least one test compound to inhibit GM3 synthase activity is evaluated, a decrease in the level of activity of this enzyme being indicative of a compound useful in the treatment and/or prevention of microvascular complications of diabetes.

Preferably, the microvascular complication of diabetes is selected from the group comprising diabetic retinopathy, diabetic nephropathy and diabetic neuropathy. Even more preferably, the microvascular complication of diabetes is diabetic nephropathy.

The test compound can be of any type. It can be a natural or synthetic compound or a mixture of such compounds. It can also be a structurally defined substance or a substance of unknown structure, for example a biological extract.

The level of GM3 synthase activity in the presence of the test compound can be compared with the control level of activity in the absence of the test compound.

In a first embodiment, the screening method comprises steps that consist in bringing at least one test compound into contact with a cell that expresses a GM3 synthase, and determining the capacity of said compound to inhibit, i.e. block or reduce, the intracellular synthesis of ganglioside GM3. A decrease in the level of synthesis of ganglioside GM3 in the cell, compared with a cell not exposed to the test compound, is indicative of a compound useful in the treatment and/or prevention of microvascular complications of diabetes.

The cell can be a cell that expresses GM3 synthase endogenously, for example a retinal pericyte or a renal mesangial cell. The cell can also be a cell transfected in a transitory or stable manner to express a GM3 synthase, with the aid of vectors for expressing the product of the GM3 synthase gene. These cells can be obtained by introducing, into prokaryotic or eukaryotic host cells, a nucleotide sequence inserted in a vector containing a sequence coding for GM3 synthase, and then culturing said cells under conditions that allow the replication and/or expression of the transfected nucleotide sequence.

The DNA vector, for example a plasmid vector, containing a sequence coding for a GM3 synthase can be introduced into a host cell by any technique known to those skilled in the art. In particular, it is possible to introduce the DNA vector in a naked form, i.e. without the aid of any kind of vehicle or system which would facilitate transfection of the vector into the cells (EP 465 529). Other available techniques are those of microinjection, electroporation, calcium phosphate precipitation or formulation with the aid of nanocapsules or liposomes. Biodegradable polyalkyl cyanoacrylate nanoparticles are particularly advantageous. In the case of liposomes, the use of cationic lipids favours encapsulation of the nucleic acids, which are negatively charged, and facilitates fusion with the negatively charged cell membranes.

Alternatively, the vector can be in the form of a recombinant virus comprising, inserted in its genome, a nucleic acid sequence coding for a GM3 synthase. The viral vector can preferably be selected from an adenovirus, a retrovirus, particularly a lentivirus, an adeno-associated virus (AAV), a herpes virus, a cytomegalovirus (CMV), a vaccine virus, etc.

The implementation of these recombinant expression techniques is well known to those skilled in the art.

Examples of host cells include especially mammalian cells such as CHO, COS-7, 293 and MDCK cells, insect cells such as SF9 cells, bacteria such as E. coli, and yeast strains.

The level of expression can be evaluated by determining the level of transcription of the gene or the level of translation of the protein encoded by this GM3 synthase gene, either directly or via e.g. a reporter gene.

The most common tests for following the transcription (i.e. determining the level of transcription) of the target gene (in this case the GM3 synthase gene) or the reporter gene are based on the northern blotting technique. The tests for following the translation (i.e. determining the level of translation) of the GM3 synthase protein or the reporter protein can be based especially on immunoassay techniques or can use fluorimetric, luminescent or other techniques for detecting reporter proteins (green fluorescent protein, GFP; luciferase; chloramphenicol acetyltransferase, CAT; etc.).

Immunoassay techniques can be carried out according to various formats well known to those skilled in the art, for example by ELISA, radioimmunoassay, in situ immunoassays, western blotting, immunofluorescence, etc. The anti-GM3 synthase protein antibodies useful for detecting the GM3 synthase protein can be produced as described below.

In another embodiment, the screening method comprises steps that consist in bringing at least one test compound into contact with a natural, mutated or recombinant GM3 synthase or a GM3 synthase of biological origin, and determining the capacity of said compound to inhibit, i.e. block or reduce, the transfer of a sialic acid residue from a sialic acid donor to a 3-hydroxyl group of a galactose residue of a sialic acid acceptor. A decrease in the level of sialic acid transfer activity in the presence of said compound, compared with the level of sialic acid transfer in the absence of said compound, is indicative of a compound that inhibits GM3 synthase and is useful in the treatment and/or prevention of microvascular complications of diabetes.

The level of GM3 synthase activity is advantageously evaluated by bringing a GM3 synthase into contact with a sialic acid donor and a sialic acid acceptor under appropriate conditions that allow the transfer of sialic acid from donor to acceptor. In general, “appropriate conditions” denote a reaction medium in which the catalytic reaction can occur. The medium can include e.g. buffers, oxidising and/or reducing agents and cofactors. The pH, temperature and ionic concentration of the medium are generally adjusted. Preferably, the GM3 synthase activity is evaluated in a medium buffered to a pH of between 6 and 7 and preferably of between 6.5 and 6.7, at a temperature of between 35 and 39° C. and preferably of 37° C. The reaction is advantageously performed in a medium containing 10 mM Mn²⁺, for example 10 mM MnCl₂. GM3 synthase activity tests have been described especially in international patent application WO 97/47749, U.S. Pat. No. 6,555,371 or the article by Wakarchuk et al. (1996).

Preferably, the GM3 synthase activity is evaluated by quantifying the transfer of sialic acid from a sialic acid donor, such as CMP-N-acetylneuraminate, to the 3-hydroxyl group of a galactose residue of lactosylceramide (LacCer) to form ganglioside GM3. A decrease in the disappearance of CMP-N-acetylneuraminate and/or lactosylceramide and/or in the formation of GM3 is indicative of a compound that inhibits GM3 synthase. A method according to the invention therefore comprises determining the level of sialic acid transfer from CMP-N-acetyl-neuraminate to lactosylceramide in the presence or absence of the test compound.

Advantageously, the sialic acid donor and/or acceptor are labelled in a detectable manner. The labelling can be effected by any appropriate technique well known to those skilled in the art. This can be e.g. radioactive, enzymatic, luminescent or fluorescent labelling or a combination of these techniques.

A preferred screening test according to the invention is described in FIG. 7. In this test, GM3 synthase is brought into contact with [¹⁴C]-CMP-sialic acid and with lactosylceramide coupled to biotin. SPA® technology (Amersham Biosciences) is based on the emission of β particles by the disintegration of certain radioactive elements. If the radioactive molecule is sufficiently close to an SPA scintillation bead, the radioactive disintegration stimulates the group of scintillants in the bead, producing a luminescent emission. The signal can be detected by a scintillation counter and/or a CCD imager. On the other hand, in the case of a free radioactive molecule in a solution containing SPA beads, i.e. a radioactive molecule that does not interact with SPA beads, the β emission associated with the disintegration of the radioactive molecule does not have sufficient energy to reach an SPA bead and no light emission is generated. In the context of the present test, measurement of the luminescent signal therefore reflects the amount of GM3 since, in the reaction medium, only this compound is associated with SPA beads via the biotin/streptavidin complex and carries a radioactive sialic acid group.

The Examples and Figures which follow illustrate the invention without implying a limitation.

### FIGURES

FIG. 1 shows the inhibition of the proliferation of pericytes (BRP) and renal mesangial cells (RMC) by AGE. The cells were exposed to 3 μM BSA or AGE for 4 days (RMC) or 7 days (BRP). The cells were then trypsinised and counted using a haemocytometer, and the total amount of proteins was determined. The results are shown as a percentage of the BSA control and represent the mean±SEM of 6 (BRP) or 9 (RMC) independent experiments, each performed in duplicate. *p<0.05 against the BSA control.

FIG. 2 shows the ganglioside biosynthesis route modified on the basis of the article by van Echten et al. (1993). Only series a and b gangliosides are detected in the BRP and RMC (surrounded gangliosides).

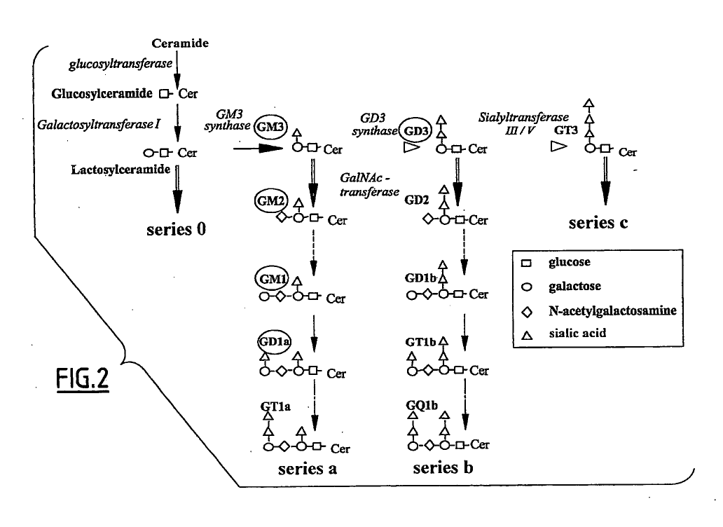

FIG. 3 illustrates the modulation of the ganglioside profile in pericytes and mesangial cells. Pericytes (A) or mesangial cells (B) were exposed to 3 μM BSA or AGE for 4 or 7 days, respectively, and then harvested. The gangliosides were extracted, purified, analysed by HPTLC and developed by staining with resorcinol, as described in the section “Materials and methods”. The results are expressed as a percentage of the BSA control and represent the mean±SEM of 6 (BRP) or 9 (RMC) independent experiments, each performed in duplicate. *p<0.05 against BSA control. In (C) and (D) the gangliosides were metabolically labelled with 1 μCi/ml of [¹⁴C]-galactose, extracted, separated by HPTLC and analysed by autoradiography. The results are expressed as a percentage of the BSA control and represent the mean±SEM of three independent experiments.

FIG. 4 shows the increase in GM3 synthase activity in isolated glomeruli and cells caused by AGE. (A) The cells were treated with 3 μM BSA or AGE for 4 days (RMC) or 7 days (BRP). The GM3 synthase activity was measured on the cell homogenates as described in the section “Materials and methods”. The control activity was 2.7 and 5.1 pmol/h/mg of proteins in the BRP and RMC, respectively. The results are expressed as a percentage of the BSA control and represent the mean±SEM of 4 or 5 independent experiments. (B) The GM3 synthase activity was measured on the highly purified homogenates of glomeruli of control mice (db/m) and db/db mice. The control activity was 4.7 pmol/h/mg of proteins. The results are expressed as a percentage of the control and represent the mean±SEM of 4-5 animals. *p<0.05 against BSA control or control mice.

FIG. 5 illustrates the inhibition of the cell proliferation caused by exogenous series a gangliosides. Pericytes (A) or mesangial cells (B) were treated with ganglioside-BSA complexes for 4 or 7 days, respectively. At the end of the treatment, the total proteins were measured. The results are expressed as a percentage of the BSA control and represent the mean±SEM of 5-6 independent experiments, each performed in triplicate. *p<0.05 against BSA control.

FIG. 6 shows that anti-series a ganglioside GM2 and GM1 antibodies protect against the effects of AGE. Pericytes (A) or mesangial cells (B) were treated with 3 μM BSA or AGE in the presence or absence of 5 μg per well of anti-GM2 or anti-GM1 polyclonal antibodies. At the end of the treatment, the cells were washed and lysed and the total amount of proteins was measured. The results are expressed as a percentage of the BSA control and represent the mean±SEM of 5-6 independent experiments, each performed in triplicate. *p<0.05 against the cells treated with AGE.

FIG. 7 illustrates the detection of GM3 synthase activity by a test which combines detection of the reaction product, ganglioside GM3, by scintigraphy and luminescence. GM3 synthase catalyses the transfer of labelled sialic acid from [¹⁴C]-CMP-sialic acid to lactosylceramide coupled to biotin (Biotin-LacCer). SPA (Scintillation Proximity Assay, Amersham Biosciences) beads coupled to streptavidin are then brought into contact with the resulting ganglioside GM3 coupled to biotin and labelled with ¹⁴C. The SPA signal resulting from the interaction between the GM3 radioactivity and the SPA beads is then measured.

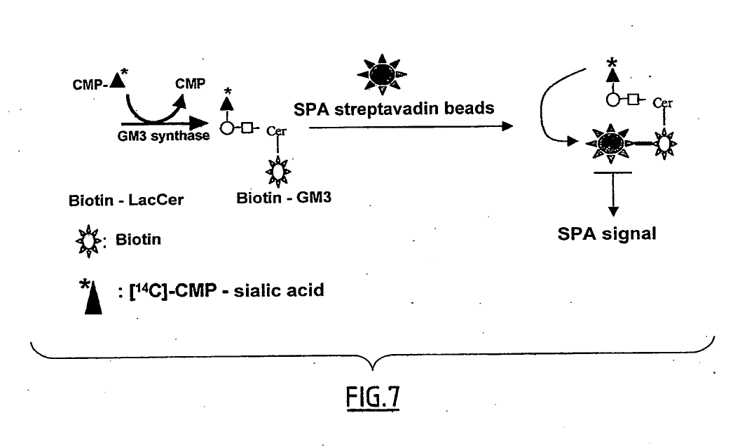

FIG. 8 shows that transfection with GM3 synthase siRNA protects RMC. 24 hours after the transfection of RMC with 400 nM GM3 synthase siRNA, the cells were treated with 3 μM BSA control or AGE. At the end of the treatment, the total proteins were measured. The results are expressed as a percentage of the BSA control and represent the mean±SEM of 6 independent experiments. *p<0.05 against the cells treated with AGE.

FIG. 9 illustrates that GM3 and GD3 synthase activities and GM3 levels are modulated in the diabetic mouse renal cortex. The GM3 synthase activity (A) and GD3 synthase activity (B) were measured on homogenates of the renal cortex of control mice (db/m) and diabetic mice (db/db). The GM3 levels (C) in control mice (db/m) and diabetic mice (db/db) are shown. In the control mice, the GM3 levels were 66±9 ng/ml of proteins. The results are expressed as a percentage of the BSA control and represent the mean±SEM of 4-6 animals. *p<0.05 against the cells treated with AGE.

### EXAMPLES

### Example 1

### Materials and Methods

Cell Isolation and Culture

Bovine retinal pericytes (BRP) were isolated from bovine retinal micro-vessels as described previously (Lecomte et al., 1996). Briefly, the retinas were obtained by dissection under sterile conditions from enucleated bovine eyes obtained from a local abattoir. After the contaminating pigmented epithelial cells of the retina had been removed, the retinas (2 per culture dish) were sliced into small pieces and homogenised in a Dounce homogeniser in a Hanks balanced saline solution (Ca²⁺/Mg²⁺-free oxygenated HBSS supplemented with Hepes 10 mM, pH 7.4, antibiotics 1%, bovine serum albumin (BSA, Sigma, Saint-Quentin Fallavier, France) 0.5%). The homogenates were centrifuged at 1000 g for 5 minutes at 4° C. and the residues were resuspended in an enzymatic solution containing collagenase/dispase (Roche Diagnostics, Mannheim, Germany) (1 mg/ml in a Ca²⁺/Mg²⁺-free oxygenated HBSS, Hepes 10 mM, pH 7.4, antibiotics 1%, DNase 20, U/ml and Nα-tosyllysine chloromethyl ketone (TLCK) 150 ng/ml, Sigma). After digestion (20 minutes at 37° C.), the microvessel fragments were placed on a 40 μm nylon filter and deposited in 6 cm dishes covered with fibronectin. The primary cultures were cultivated in a DMEM (Dulbecco modified Eagle's medium) supplemented with 10% of foetal bovine serum (Gibco, Invitrogen Corporation, N.Y., United States), 1% of glutamine and 1% of penicillin/streptomycin (Sigma), and the medium was replaced every two days. After the adhesion period, an excrescence of pericytes from the microvessels occurred after 48 hours and the cells reached confluence in about 10 days. The BRP cultures were 100% pure, as characterised by their polygonal irregular morphology with pseudopodia and their growth into non-apposed cells, and as evaluated by positive labelling for both α₁-actin and a specific glycolipid antigen (3G5 antibody) and by negative labelling for the von Willebrand factor expressed in the endothelial cells (Lecomte et al., 1996). The cells were passaged with trypsin-EDTA (Sigma) (1:3) and culture was continued with the same medium up to the second passage, during which the BRP were treated.

Rat renal mesangial cells (RMC) were obtained from purified glomeruli of young male Wistar rats (Charles River, l'Arbresle, France). Briefly, cortex fragments were isolated from rat kidneys freshly removed under sterile conditions. Small pieces were mechanically forced through a 230 μm mesh with HBSS buffer. The glomeruli, passed through this mesh, were then forced through a 73.7 μm mesh. Finally, they were placed on a 70 μm mesh and placed in 6 cm dishes covered with fibronectin (4 dishes for 2 renal glomeruli) in DMEM supplemented with 20% of foetal bovine serum, 1% of glutamine and 1% of penicillin/streptomycin. After the attachment period, the excrescence of RMC from glomeruli occurred after three weeks and the cells were then cultivated to the fifth passage in order to remove the residual epithelial and endothelial cells. The RMC are characterised by morphological criteria (star shape, wick shape when they are confluent) and by positive labelling with vimentin, smooth muscle α-actin and Thy-1 antigen. The cells were then cultivated in DMEM supplemented with 15% of foetal calf serum, 1% of glutamine and 1% of penicillin/streptomycin. They were used between the fifth and fifteenth passages.

Isolation of Mouse Glomeruli

Highly purified glomeruli were obtained from diabetic (db/db) or control (db/m) eleven-week-old mice (Charles River) by the magnetic bead perfusion technique, as described by Takemoto et al. (Takemoto et al., 2002). Briefly, the mice were perfused through the heart with a Dynabeads solution (Dynal, Compiègne, France) and the kidneys were then removed, finely sliced and digested with collagenase (Roche Diagnostics). After filtration, the glomeruli which had accumulated beads in their capillaries were retained by magnetism and then washed twice before homogenisation. This technique provides pure preparations of glomeruli with a low degree of tissue contamination.

Isolation of Mouse Renal Cortices

Renal cortex fragments were isolated from the kidneys of diabetic (db/db) or control (db/m) eleven-week-old mice (Charles River). Briefly, the animals were anaesthetised and sacrificed and the kidneys were removed. The renal cortex fragments were then obtained by dissection and mechanically homogenised with a Dounce homogeniser in a 25 mM Hepes buffer containing 1 mM EDTA and 10 μl/ml of protease inhibitors.

Preparation of AGE

AGE were prepared by incubating bovine serum albumin (final concentration of 7.2 mg/ml) (Sigma) with 100 mM methylglyoxal (Sigma) at 37° C. for 50 hours. Bovine serum albumin (BSA) was incubated under the same conditions in the absence of methylglyoxal and used as a control preparation (BSA control). The AGE and the BSA control were purified on PD10 Sephadex G25 columns (Amersham Biosciences, Uppsala, Sweden) to remove the salts and unreacted carbonyls, and were then sterilised by filtration and kept at −20° C. until used.

Treatment with AGE

The AGE and the BSA control (final concentration of 3 μM) were added to the culture medium 24 hours after inoculation. Each cell type was treated for one passage (about 7 days for the BRP and 4 days for the RMC). The culture medium was replaced with a fresh medium every two days.

Measurement of Cell Growth

At the end of the treatment, the cells were harvested with trypsin and the cell residues were washed twice with a phosphate buffered saline solution (iced PBS) (Sigma). For each sample, one aliquot of cells was counted using a haemocytometer in order to determine the number of cells. Another aliquot was used to measure the proteins by the Bradford technique.

Analysis of Gangliosides

For metabolic labelling of the gangliosides, 0.2 μCi/ml or 1 μCi/ml of [¹⁴C(U)]-D-galactose (329.5 mCi/mmol) (PerkinElmer Life Sciences, Boston, Mass.) was added to the culture medium overnight for the labelling experiments with a high specific activity (GM2 and GM1 measuring experiment). The cells (5-8×10⁵ pericytes or 12-20×10⁵ mesangial cells) were then harvested by trypsinisation and washed three times in PBS. The gangliosides were extracted from the cell residues by the method described by Bouchon et al. (Bouchon et al., 1990), modified by Natalizio et al. (Natalizio et al., 2002). Briefly, the cell residues were dispersed in 2 ml of chloroform (C)/methanol (M) (1:1, v/v), mixed vigorously and extracted overnight at 4° C. After centrifugation, the residues were extracted twice with 2 ml of the same solvent. The pooled extracts of total lipids were dried by evaporation and separated with the aid of a 1 mM C/M/PBS solution (10:10:7, v/v/v). The upper phases, containing the gangliosides, were then desalted on a C18 silica gel column (Waters Corporation, Milford, Mass.) and analysed by HPTLC (Merck, Darnstadt, Germany). The plates were developed in 0.2% C/M/CaCl₂ (55:45:10, v/v/v). The gangliosides were visualised by autoradiography using a phosphorus screen and a Storm 820 (Molecular Dynamics, Amersham Pharmacia Biotech, Piscataway, United States) and by labelling with resorcinol (specific stain for gangliosides: resorcinol 0.3% (Sigma), CuSO₄ 0.03%, HCl 30%) using an Image Master VDS-CL (Amersham Pharmacia Biotech). Quantification was performed using an Image Quant (Molecular Dynamics). As GT1b was absent from the ganglioside profiles of both the BRP and the RMC, it was added to the samples as an internal standard prior to lipid extraction.

Measurement of GM3 Synthase Activity

At the end of the treatment, the cells were washed with PBS, incubated for 20 minutes at 4° C. in 50 μl of lysis buffer (20 mM sodium cacodylate, pH 6.6 (Sigma), 0.2% of Triton X-100, 1 mM EDTA, 10 μl/ml of protease inhibitors, Calbiochem, La Jolla, Calif., United States) and then collected by scraping. Four dishes of BRP (about 2-3×10⁶ cells) and three dishes of RMC (about 3-6×10⁶ cells) were pooled. The cell lysates were centrifuged at 10,000 g for 5 minutes and the proteins in the supernatants were used to measure the GM3 synthase activity. The glomerular residues were mechanically homogenised with a syringe in 25 mM Hepes containing 1 mM EDTA and 10 μl/ml of protease inhibitors. The homogenates were then centrifuged at 1000 g for 2 minutes and the postnuclear supernatant was used to measure the GM3 synthase activity. Equivalent amounts of proteins for each sample (about 500 μg for the cells and 100 μg for the glomeruli) were used to perform the test. The samples were mixed with an equal volume of reaction buffer containing final concentrations of 0.1 mM lactosylceramide (Matreya, Biovalley, Marne la Vallée, France), 4 μCi/ml of [sialic-4,5,6,7,8,9-¹⁴C]-CMP-sialic acid (325.2 mCi/mmol) (PerkinElmer Life Sciences), 100 μM CMP-sialic acid (Sigma), 10 mM MgCl₂, 0.2% of Triton X-100 and 100 mM sodium cacodylate, pH 6.6. After agitation, the reaction mixtures were incubated at 37° C. for 50 minutes. The reactions were stopped by loading the samples onto silica gel 60 columns (Merck) to separate the excess substrates from the products. After the columns had been washed with water, the gangliosides were eluted with C/M (1:1, v/v) and the solvent was dried under nitrogen. Finally, the gangliosides were separated by thin layer chromatography (Merck) and the reaction products were developed by autoradiography. The GM3 synthase activity was expressed in pmol of GM3 produced/h/mg of proteins.

Treatment with Exogenous Gangliosides

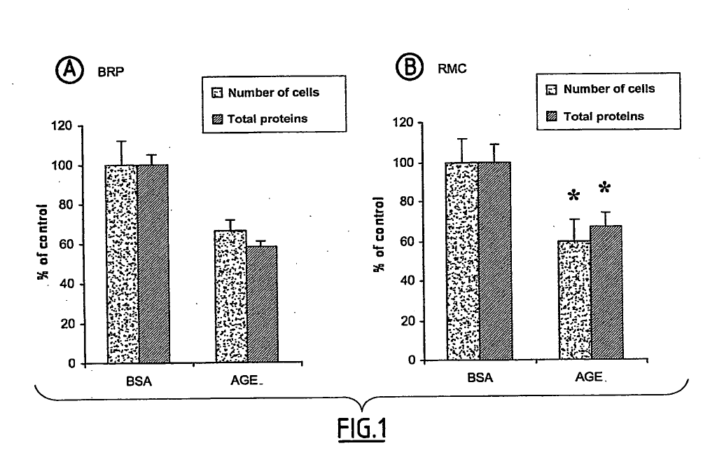

To evaluate the effect of exogenous gangliosides on the proliferation of BRP and RMC, the cells were cultivated in 96-well plates. Exogenous glycolipids GM3, GM2, GM1 and GD1a, glucosylceramide and lactosylceramide (Matreya) were added to the complete culture medium in a final concentration of 50 μM in the form of complexes with BSA in a 1:1 ratio in DMEM/10 mM Hepes, pH 7.4. At the end of the treatment, the cells were washed twice with PBS and lysed for 30 minutes at 37° C. in 50 μl of a Ripa lysis buffer (PBS 10 mM, NP40 1% (Pierce, Perbio Science, Brebières, France), sodium deoxycholate 0.5%, SDS 0.1%, protease inhibitors 10 μl/ml). As the number of cells in our experiments was correlated with the total protein concentrations (cf. FIG. 1), the total proteins were measured using the BCA protein test (Pierce) in order to evaluate the cell proliferation.

Treatment with Anti-Series a Ganglioside Antibodies

To block the potential effects of series a gangliosides, the cells were cultivated in 96-well plates and treated with 3 μM AGE or control BSA in the presence or absence of 50 μg/ml of anti-GM2 polyclonal antibody (Calbiochem) or anti-GM1 polyclonal antibody (Matreya). At the end of the treatment, the cells were washed twice with PBS and lysed in 50 μl of Ripa lysis buffer and the proteins were measured in order to quantify the cell proliferation.

Transfection of RMC with a GM3 Synthase siRNA

To block the GM3 synthase activity, RMC were cultivated in 6-well culture plates up to 30% confluence and then transfected with an interfering RNA (siRNA) specific for GM3 synthase, which was designed against a rat cDNA sequence (Ambion, Huntingdon, UK) using an Oligofectamine reagent (Invitrogen Corporation). The GM3 synthase antisense sequence used was GGGUUAUUCUGAACAUGUUtt (SEQ ID No: 5). In preliminary experiments, a dose-response study was carried out by transfecting the cells with increasing concentrations of siRNA (0-800 nM). After 72 h of transfection, the GM3 synthase activity was measured on homogenates of transfected cells. The proliferation was then evaluated on the cells transfected with the siRNA. For this purpose, 24 hours after transfection with 400 nM siRNA, the cells were treated with 3 μM BSA control or AGE (3 days). The cells were then washed twice with PBS and lysed in a Ripa lysis buffer and the proteins were measured in order to evaluate the cell proliferation.

Statistical Analysis

The data are expressed as means±SEM and presented as a percentage of the controls. In the cell studies, the Wilcoxon rank test was used to evaluate the significance of the difference between the groups. The Student t-test was used for the GM3 synthase activity in the experiments on mice. p<0.05 was considered to be statistically significant.

### Example 2

### Results

1—AGE Inhibit the Proliferation of Pericytes and Mesangial Cells

To compare the effect of AGE on the proliferation of pericytes and mesangial cells, the cells were treated with BSA or AGE at a concentration of 3 μM for 4 to 7 days. The cell counts showed that AGE reduced the number of pericytes and mesangial cells by 33 and 40%, respectively (FIG. 1). The total proteins were also measured and were found to have decreased in correlation with the number of cells. These results demonstrate that AGE have similar adverse effects on the proliferation of both pericytes and mesangial cells and enabled the inventors to elucidate the common mechanisms involved in the response to AGE in both these cell types.

2—AGE Increase Series a Gangliosides in Pericytes and Mesangial Cells

The inventors' previous results suggested that AGE could modulate the ganglioside profile in retinal microvascular cells (Natalizio et al., 2001). The ganglioside profiles were analysed in BRP and RMC in response to the BSA control or the AGE. The ganglioside profile is specific for the cell type. Under control conditions, the main gangliosides in the pericytes were series a gangliosides GM3 (63% of the total gangliosides detected) and GM1 (9%) and series b ganglioside GD3 (28%). The profile in the mesangial cells differed by the very small amount of GD3 (5%) and, in the case of series a, by the presence of GD1a (20%), GM3 still being the main ganglioside (75%).

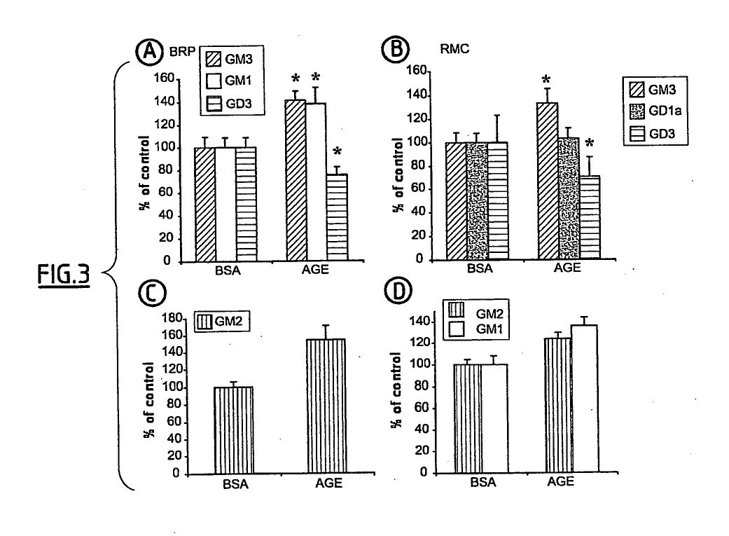

An increase in series a gangliosides and a decrease in series b gangliosides were observed in both cell types treated with AGE (FIG. 3). In the pericytes, series a gangliosides GM3 and GM1 were increased by about 40%, whereas series b ganglioside GD3 was reduced by 24% (FIG. 3A). In the mesangial cells, GM3 was increased by 33%, whereas GD3 was reduced by 30%; the GD1a levels were not affected (FIG. 3B). Similar results were obtained by autoradiography after labelling with galactose. As series a gangliosides GM2 in the BRP and GM2 and GM1 in the RMC were difficult to detect by labelling with resorcinol, the cells were labelled with [¹⁴C]-D-galactose of high specific activity and the gangliosides were then analysed in the control cells and the cells treated with AGE. The results showed that GM2 was increased by 55% in the BRP (FIG. 3C) and that GM2 and GM1 were increased by 25 to 35% in the RMC (FIG. 3D). These results indicated that AGE induce similar modifications to the ganglioside profiles in both pericytes and mesangial cells. They also suggest that the increase in series a gangliosides may be a common mechanism underlying the decrease in cell proliferation.

3—AGE Increase GM3 Synthase Activity in Pericytes and Mesangial Cells

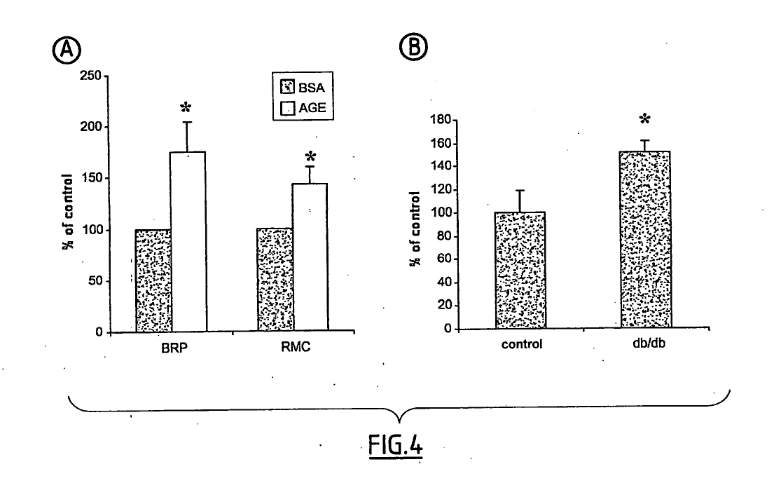

In an attempt to explain the mechanism responsible for the observed increase in series a gangliosides, the activity of GM3 synthase, the limiting enzyme for the synthesis of series a gangliosides, was measured in the control and treated cells. The results presented in FIG. 4 show that the treatment with AGE increased the GM3 synthase activity by a factor of about 1.8 in the pericytes and of about 1.5 in the mesangial cells, very probably by increasing the maximum rate of the enzymatic reaction. These results suggest that AGE exert common mechanisms in RMC and BRP by regulating the GM3 synthase.

4—Exogenous Series a Gangliosides Inhibit the Proliferation of Pericytes and Mesangial Cells

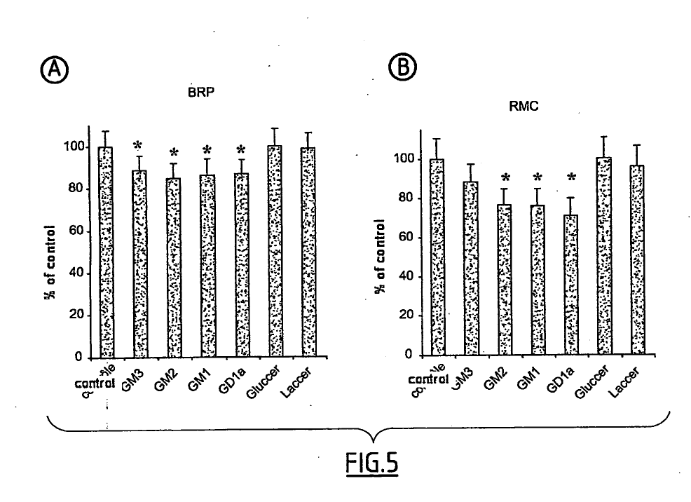

The inventors studied whether the exogenous addition of series a gangliosides would affect the proliferation of pericytes and mesangial cells. The cells were treated for one passage (7 days for the BRP and 4 days for the RMC) with 50 μM gangliosides and, as control, with the non-sialylated glucosylceramide and lactosylceramide precursors. FIG. 5 shows that GM2, GM1 and GD1a inhibited the proliferation of the pericytes and mesangial cells most effectively (about 15 to 30%). GM3 weakly inhibited the proliferation of the pericytes, but had no significant effect on the mesangial cells. Glucosylceramide and lactosylceramide, used as control, had no effect. These results indicate that series a gangliosides inhibit the proliferation of pericytes and mesangial cells. In particular, GM2 and GM1 are increased in BRP and RMC in response to AGE and they degrade the proliferation of both these cell types; these series a gangliosides could therefore be the common mediators of the AGE effect.

5—Anti-Series a Ganglioside Antibodies Protect Pericytes and Mesangial Cells Against Inhibition of the Proliferation Caused by AGE

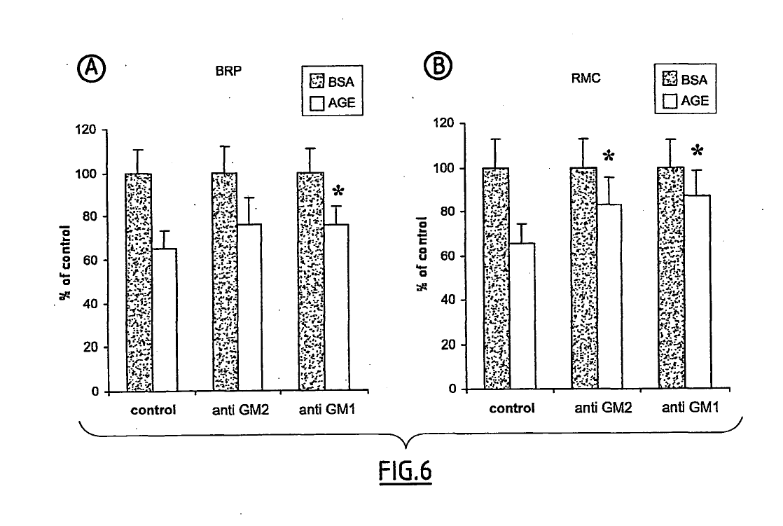

To study whether GM2 and GM1 mediate the effects of AGE, the cells were treated with AGE in the presence of anti-GM1 and anti-GM2 antibodies. In the control cells treated with BSA, the proliferation did not differ in the presence or absence of the anti-ganglioside antibodies. Treatment of the cells with AGE in the presence of anti-GM2 and anti-GM1 antibodies partially prevented the decrease in the proliferation of pericytes and mesangial cells (FIG. 6). In the light of the effects of exogenous gangliosides, these observations suggest that series a gangliosides, particularly GM1 and GM2, are the common mediators of the inhibition of the proliferation induced by AGE in pericytes and mesangial cells.

6—GM3 Synthase Activity is Increased in Diabetic Mouse Glomeruli

To evaluate the effect of a diabetic environment on GM3 synthase activity in vivo, the enzymatic activity was measured in highly purified glomeruli of a db/db diabetic mouse model. The results presented in FIG. 4B show that the GM3 synthase activity was increased by 50% in the glomeruli of diabetic cells, compared with the controls (db/m).

7—A GM3 Synthase siRNA Partially Protects from Effects Induced by AGE

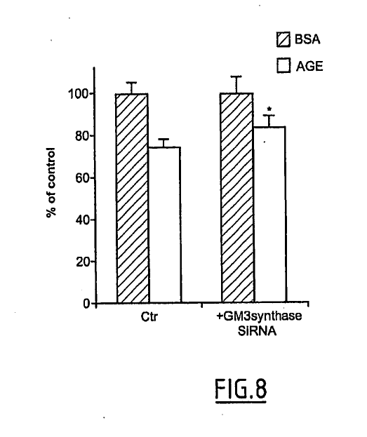

To elucidate in greater detail the role of series a gangliosides in the mediation of AGE effects, RMC were transfected with a GM3 synthase siRNA. Preliminary experiments showed that the siRNA effectively inhibited GM3 synthase activity in the RMC. The proliferation of the transfected cells and treated cells was then measured. As shown in FIG. 8, the effects of AGE on the proliferation of the RMC are partially inhibited in the cells transfected with the GM3 synthase siRNA. These results clearly demonstrate the involvement of gangliosides in the mediation of AGE effects. The partial nature of the effects can be explained by the fact that: (i) the AGE effects were less potent than in the previous experiments, given that the cells were treated to a more advanced level of confluence so as to increase the efficacy of transfection, and (ii) the GM3 synthase activity was inhibited by only 50%.

8—GM3 and GD3 Synthase Activities and GM3 Levels are Modified in Diabetic Mouse Renal Cortex

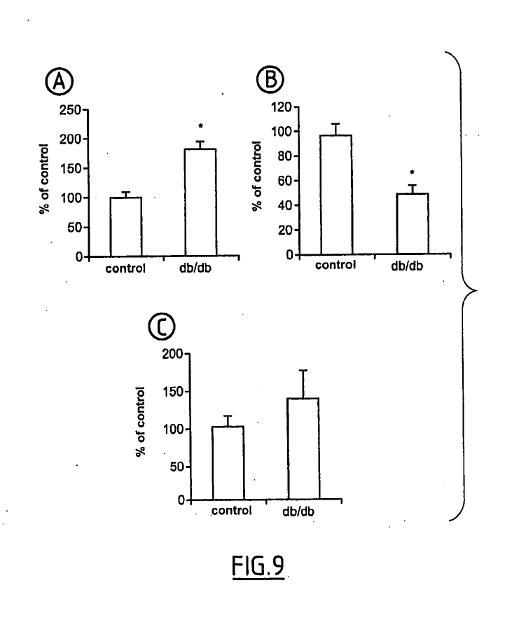

To evaluate ex vivo the effect of a diabetic environment on the ganglioside biosynthesis pathway, the GM3 and GD3 synthase activities were measured on homogenates of renal cortex of the db/db diabetic mouse model. The results presented in FIGS. 9A-B show that the GM3 synthase activity was increased by 80%, whereas the GD3 synthase activity was reduced by 50% in the diabetic mouse renal cortices, compared with the controls (db/m). The gangliosides were also analysed and showed an increase, albeit not statistically significant, in the GM3 levels in the renal cortex of db/db diabetic mice (FIG. 9C). Overall, these results therefore demonstrate the physiopathological sense of the results obtained in RMC and BRP treated with AGE.

### REFERENCES

Bass, Cell, 2000, 101, 235-238

Benoit et al. (1982) PNAS USA, 79, 917-921

Bouchon et al. (1990) Biochim. Biophys. Acta, 1051, 1-5

Cech T. R. (1989) RNA as an enzyme. Biochem. Int., 18, 7-14

Feigner P. L., Ringold G. M. (1989) Cationic liposome-mediated transfection. Nature, 337, 387-8

Fong et al. (2003) Diabetes Care, 26, 226-229

Forrester et al. (1997) Pathogenesis of diabetic retinopathy and cataract. In Textbook of diabetes. Pickup J., Williams G., Eds Oxford, 45.1-45.19

Fraley R., Subramani S., Berg P., Papahadjopoulos D. (1980) Introduction of liposomeencapsulated SV40 DNA into cells. J. Biol. Chem., 255,10431-5

Harlow E. et al. (1988) Antibodies: A Laboratory Manual, Cold Spring Harbor Laboratory, New York

Hakomori et al. (1990) J. Biol. Chem., 265, 18713-18716

Hatanaka Y. et al. (1996) Synthesis and characterisation of a carbene-generating biotinylated lactosylceramide analog as a novel chromogenic photo-probe for GM3 synthase. Chem. Pharm. Bull. (Tokyo), 44(5), 1111-4

Hofmann et al. (1999) Cell, 97, 889-901

Inokuchi J. et al. (1998) A synthetic ceramide analog (L-PDMP) up-regulates neuronal function. Ann. NY Acad. Sci., 845, 219-24

Jayasena (1999) Aptamers: an emerging class of molecules that rival antibodies in diagnostics. Clin. Chem., 45(9), 1628-50

Kaneda Y., Iwai K., Uchida T. (1989) Increased expression of DNA co-introduced with nuclear protein in adult rat liver. Science, 243, 375-8

Krolewski et al. (1997) Clinical features and epidemiology of diabetic nephropathy. In Textbook of diabetes, Pickup J., Williams G., Eds Oxford, 53.1-53.13

Köhler and Milstein (1975) Nature, 256, 495-497

Lal et al. (2002) Kidney Int., 61, 2006-2014

Lecomte et al. (1996) J. Neurochem., 66, 2160-2167

Marks et al. (1991) J. Mol. Biol., 222, 581-597

Natalizio et al. (2001) Biochem. Biophys. Res. Commun., 281, 78-83

Pagano J. S., McCutchan J. H., Vaheri A. (1967) Factors influencing the enhancement of the infectivity of poliovirus ribonucleic acid by diethylaminoethyl-dextran. J. Virol., 1, 891-7

Rippin J. D., Barnett A. H., Bain S. C. (2004) Cost-effective strategies in the prevention of diabetic nephropathy. Pharmacoeconomics, 22(1), 9-28

Rubin-Carrez C. (2000). Les mimes peptidiques (Peptide mimics). Le technoscope, Biofutur, vol. 199

Sambrook, Fritsch & Maniatis, Molecular Cloning: A Laboratory Manual, Second Edition (1989) Cold Spring Harbor Laboratory Press, Cold Spring Harbor, N.Y.

Schmidt et al. (1994) Arterioscler. Thromb., 14, 1521-1528

Schmidt et al. (1995) J. Clin. Invest., 96, 1395-1403

Sharp, Genes Dev., 2001, 15, 485, 49

Singh et al. (2001) Diabetologia, 44, 129-146

Scott J. K. and Smith G. P., Science, 1990, 249, 386-390

Stitt et al. (2003) Exp. Mol. Pathol., 75, 95-108

Takemoto et al. (2002) Am. J. Pathol., 161, 799-805

Tuerk C. and Gold L. (1990) Systematic evolution of ligands by exponential enrichment: RNA ligands to bacteriophage T4 DNA polymerase. Science, 3, 249, 505-10

Van Echten et al. (1993) J. Biol. Chem., 268, 5341-5344

Wakarchuk et al. (1996) Functional relationships of the genetic locus encoding the glycosyltransferase enzymes involved in expression of the lacto-N-neotetraose terminal lipopolysaccharide structure in Neisseria meningitidis. J. Biol. Chem., 271(32), 19166-73

Wautier et al. (2001) Diabetes Metab., 27, 535-542

Wolf et al. (2000) Kidney Int. Suppl., 77, S59-S66

1. Use of a GM3 synthase inhibitor for the manufacture of a drug intended for the treatment of a microvascular complication of diabetes.

2. Use according to claim 1 wherein the microvascular complication of diabetes is diabetic nephropathy.

3. Use according to claim 1 or 2 wherein the inhibitor is an inhibitor of the expression of the GM3 synthase gene or protein.

4. Use according to claim 3 wherein the inhibitor is selected from the group comprising antisense nucleic acids, ribozymes, interfering RNAs and aptamers.

5. Use according to claim 4 wherein the inhibitor is an antisense nucleic acid sequence that hybridises specifically with the sequence SEQ ID No: 1 under highly stringent conditions.

6. Use according to claim 1 or 2 wherein the inhibitor is an inhibitor of GM3 synthase activity.

7. Use according to claim 6 wherein the inhibitor is a monoclonal or polyclonal antibody directed against human GM3 synthase.

8. In vitro method of screening or identifying compounds useful in the treatment and/or prevention of microvascular complications of diabetes, wherein the capacity of at least one test compound to inhibit GM3 synthase activity is evaluated, a decrease in the level of GM3 synthase activity being indicative of a compound useful in the treatment and/or prevention of microvascular complications of diabetes.

9. Method according to claim 8 wherein the microvascular complication of diabetes is diabetic nephropathy.

10. Method according to claim 8 or 9, said screening method comprising steps that consist in bringing at least one test compound into contact with a cell that expresses a GM3 synthase, and determining the capacity of said compound to inhibit the intracellular synthesis of ganglioside GM3, a decrease in the level of synthesis of ganglioside GM3 in the cell being indicative of a compound useful in the treatment and/or prevention of microvascular complications of diabetes.

11. Method according to any one of claims 8 to 10, said screening method comprising steps that consist in bringing at least one test compound into contact with a GM3 synthase and determining the capacity of said compound to inhibit the transfer of a sialic acid residue from a sialic acid donor to a 3-hydroxyl group of a galactose residue of a sialic acid acceptor, a decrease in the level of sialic acid transfer activity being indicative of a compound that inhibits GM3 synthase and is useful in the treatment and/or prevention of microvascular complications of diabetes.

12. Method according to claim 11 wherein the level of sialic acid transfer from CMP-N-acetylneuraminate to lactosylceramide is determined.

13. Method according to claim 11 or 12 wherein the sialic acid donor and/or acceptor are labelled in a detectable manner.


SEQUENCE LISTING (5 sequences)

SEQ ID NO: 1 - DNA, 2362 bp, Homo sapiens
  CDS (278)..(1366)
ctgagcgggggagcggcggcccccagctgaatgggcgcgagagcggcgctgggggcgggtgggggcgcggggtaccgggctggcggccggccggcgccccctcattagtatgcggacgaaggcggcgggctgcgcggagcggcgtcccctgcagccgcggaccgaggcagcggcggcacctgccggccgagcaatgccaagtgagtacacctatgtgaaactgagaagtgattgctcgaggccttccctgcaatggtacacccgagctcaaagcaagatgagaaggcccagcttgttattaaaagacatcctcaaatgtacattgcttgtgtttggagtgtggatcctttatatcctcaagttaaattatactactgaagaatgtgacatgaaaaaaatgcattatgtggaccctgaccatgtaaagagagctcagaaatatgctcagcaagtcttgcagaaggaatgtcgtcccaagtttgccaagacatcaatggcgctgttatttgagcacaggtatagcgtggacttactcccttttgtgcagaaggcccccaaagacagtgaagctgagtccaagtacgatcctccttttgggttccggaagttctccagtaaagtccagaccctcttggaactcttgccagagcacgacctccctgaacacttgaaagccaagacctgtcggcgctgtgtggttattggaagcggaggaatactgcacggattagaactgggccacaccctgaaccagttcgatgttgtgataaggttaaacagtgcaccagttgagggatattcagaacatgttggaaataaaactactataaggatgacttatccagagggcgcaccactgtctgaccttgaatattattccaatgacttatttgttgctgttttatttaagagtgttgatttcaactggcttcaagcaatggtaaaaaaggaaaccctgccattctgggtacgactcttcttttggaagcaggtggcagaaaaaatcccactgcagccaaaacatttcaggattttgaatccagttatcatcaaagagactgcctttgacatccttcagtactcagagcctcagtcaaggttctggggccgagataagaacgtccccacaatcggtgtcattgccgttgtcttagccacacatctgtgcgatgaagtcagtttggcgggttttggatatgacctcaatcaacccagaacacctttgcactacttcgacagtcaatgcatggctgctatgaactttcagaccatgcataatgtgacaacggaaaccaagttcctcttaaagctggtcaaagagggagtggtgaaagatctcagtggaggcattgatcgtgaattttgaacacagaaaacctcagttgaaaatgcaactctaactctgagagctgtttttgacagccttcttgatgtatttctccatcctgcagatactttgaagtgcagctcatgtttttaacttttaatttaaaaacacaaaaaaaattttagctcttcccactttttttttcctatttatttgaggtcagtgtttgtttttgcacaccattttgtaaatgaaacttaagaattgaattggaaagacttctcaaagagaattgtatgtaacgatgttgtattgatttttaagaaagtaatttaatttgtaaaacttctgctcgtttacactgcacattgaatacaggtaactaattggaaggagaggggaggtcactcttttgatggtggccctgaacctcattctggttccctgctgcgctgcttggtgtgacccacggaggatccactcccaggatgacgtgctccgtagctctgctgctgatactgggtctgcgatgcagcggcgtgaggcctgggctggttggagaaggtcacaacccttctctgttggtctgccttctgctgaaagactcgagaaccaaccagggaagctgtcctggaggtccctggtcggagagggacatagaatctgtgacctctgacaactgtgaagccaccctgggctacagaaaccacagtcttcccagcaattattacaattcttgaattccttggggattttttactgccctttcaaagcacttaagtgttagatctaacgtgttccagtgtctgtctgaggtgacttaaaaaatcagaacaaaacttctattatccagagtcatgggagagtacaccctttccaggaataatgttttgggaaacactgaaatgaaatcttcccagtattataaattgtgtatttaaaaaaaagaaacttttctgaatgcctacctggcggtgtataccaggcagtgtgccagtttaaaaagatgaaaaagaataaaaacttttgaggaac

SEQ ID NO: 2 - protein, 362 aa, Homo sapiens
MRRPSLLLKDILKCTLLVFGVWILYILKLNYTTEECDMKKMHYVDPDHVKRAQKYAQQVLQKECRPKFAKTSMALLFEHRYSVDLLPFVQKAPKDSEAESKYDPPFGFRKFSSKVQTLLELLPEHDLPEHLKAKTCRRCVVIGSGGILHGLELGHTLNQFDVVIRLNSAPVEGYSEHVGNKTTIRMTYPEGAPLSDLEYYSNDLFVAVLFKSVDFNWLQAMVKKETLPFWVRLFFWKQVAEKIPLQPKHFRILNPVIIKETAFDILQYSEPQSRFWGRDKNVPTIGVIAVVLATHLCDEVSLAGFGYDLNQPRTPLHYFDSQCMAAMNFQTMHNVTTETKFLLKLVKEGVVKDLSGGIDREF

SEQ ID NO: 3 - DNA, 2221 bp, Mus musculus
  Description of Combined DNA/RNA Molecule Synthetic oligonucleotide
  CDS (151)..(1314)
tctttgcgataccccaggcccagcggctcctccccagccctgcgacgccggacgcgcctgctaggggacacgggcggagggtcgcggcccctggctgcctacatgggcgcccccggcgagctgcgcaggtgtggacgcggcgctgcggcaatgccaagtgagttcacctctgcaaagctgagaagtgattgctcaaggacctccctgcaatggtacacccgaacccagcacaagatgagaagacccagcttgttaataaaagacatctgcaagtgcacgttggttgcatttggagtctggctcctgtacatcctcattttgaattacaccgctgaagaatgtgacatgaaaagaatgcactatgtggaccctgaccggataaagagagctcagagctatgctcaggaagtcttgcagaaggaatgtcggcccaggtacgcgaagacggctatggctctgttatttgaggacaggtacagcatcaacttggagccttttgtgcagaaggtccccacggccagtgaagctgagctcaagtatgacccgccttttggattccggaagttctccagtaaagtccagagcctcttggatatgctgcccgaacatgacttttctgaacacttgagagccaaggcctgcaagcgctgtgtggttgttgggaacgggggcatcctgcacggactagagctgggtcacgccctcaaccagttcgatgtggtaataaggttgaacagtgcgccagttgagggttactctgaacacgttgggaataaaactactataaggatgacttacccagagggtgcgccactgtcggacgttgaatactacgccaatgatttgttcgttactgttttatttaagagtgttgatttcaagtggcttcaagcaatggtaaaaaatgaaagcctgcccttttgggttcgcctcttcttttggaagcaagtggcagaaaaagtcccactccagccaaagcacttcaggattttgaacccagttatcatcaaagaaactgccttcgacatccttcagtactcagagcctcagtcaagattctggggccatgataagaacatccccacgatcggcgtcattgccgttgtcttggctacacatctgtgtgatgaagtcagcctggcaggctttggctacgacctcagtcaacccaggacccctctgcactactttgacagtcagtgcatgggcgccatgcactggcaggtcatgcacaatgtgaccacagagaccaagttcctcctgaagctcctcaaggagggcgtggtggaggacctcagcggcggcatccactgagaactcggaacacggcaaacctcacccagcaccgcagctgagagcgtggtgagcagcctccacagggacttcaccctgcagctgcttcgatgtgcagctagtgttttcaaactccacattttttttaaaaaaggaaaagaaagaacaacagcaacaacaaaagctctgctctgtgcacctcttcgtcctatttatttgaagtcagtgttggattttgcacagttttgtaagttaatcttaagaatgggattggaaggacttttcaaagagaattgtatagtttattgttttttaaggaagtaatttaatttgcagaaactgtacacacgtactctgctcaggtgttgaggtgggaggagaggggcttctggcccctggatgatggctgtgatgcccgatactggggtctgctgctctgtttggtagaactgatggcagagaaacttcctgcctccaggataaagggcttactcatcacctctggcagctgctagacaagttcataacccctttctgctagtccatctgccagctggctcgcaggactcaggcagggcagctgtcccggaggctgctggttggtgagccactgtcagctgagcgccgtgatgttgccccagggtggaagaagccacacttcctacactgtcagggcacttttaaacttctggaggggtgtgtgtgtgtgtgtgtgtgtgtgtgtgtgtgtgtgtgtgtgtgtgtgtgttcattctgcccttccaaatcatctaagtgttatttaaggcactctgctgtttgtatgagatggctcatagatattatgacaaagcctttgttatccaggccatgggaagaggaaaaagaaaagaaagagagaaaagaataaaagcttttgaggagcccctgtgatttcctgaaaaaaaaa

SEQ ID NO: 4 - protein, 387 aa, Mus musculus
MPSEFTSAKLRSDCSRTSLQWYTRTQHKMRRPSLLIKDICKCTLVAFGVWLLYILILNYTAEECDMKRMHYVDPDRIKRAQSYAQEVLQKECRPRYAKTAMALLFEDRYSINLEPFVQKVPTASEAELKYDPPFGFRKFSSKVQSLLDMLPEHDFSEHLRAKACKRCVVVGNGGILHGLELGHALNQFDVVIRLNSAPVEGYSEHVGNKTTIRMTYPEGAPLSDVEYYANDLFVTVLFKSVDFKWLQAMVKNESLPFWVRLFFWKQVAEKVPLQPKHFRILNPVIIKETAFDILQYSEPQSRFWGHDKNIPTIGVIAVVLATHLCDEVSLAGFGYDLSQPRTPLHYFDSQCMGAMHWQVMHNVTTETKFLLKLLKEGVVEDLSGGIH

SEQ ID NO: 5 - DNA, 21 bp, Artificial Sequence
  Description of Combined DNA/RNA Molecule Synthetic oligonucleotide
  Description of Artificial Sequence Synthetic oligonucleotide
ggguuauucugaacauguutt